(19) United States
(12) Patent Application Publication
(10) Pub. No.: US 2014/0212508 A1
(43) Pub. Date: Jul. 31, 2014

(54) COPPER/ZINC SUPEROXIDE DISMUTASE (SOD) FORMULATION FOR THE TREATMENT OF TRAUMAS INCLUDING AMYOTROPIC LATERAL SCLEROSIS

(71) Applicant: John Wayne Kennedy, Stevensville, MD (US)

(72) Inventor: John Wayne Kennedy, Stevensville, MD (US)

(21) Appl. No.: 14/244,084

(22) Filed: Apr. 3, 2014

Related U.S. Application Data
(63) Continuation-in-part of application No. 11/932,260, filed on Oct. 31, 2007.
Application No. 11/932,260 is a continuation-in-part of application No. 11/616,317, filed on Dec. 27, 2006, now abandoned.
Application No. 11/616,317 is a division of application No. 10/027,692, filed on Dec. 20, 2001, now Pat. No. 7,163,709.

(51) Int. Cl. A61K 33/34; A61K 33/04; A61K 33/06; A61K 33/24; A61K 33/30; A61K 33/32

(52) U.S. Cl. CPC A61K 33/34; A61K 33/30; A61K 33/32; A61K 33/06; A61K 33/24; A61K 33/04
USPC 424/630; 424/643; 424/639; 424/682; 424/646; 424/655

(57) ABSTRACT

A Copper/Zinc superoxide dismutase (Cu/Zn SOD) composition comprising a therapeutically effective amount of ionic mineral(s) in a ligand complex and a pharmaceutically acceptable carrier for the neutralization of superoxide produced trauma and/or stress-related conditions including, but not limited to, conditions such as myotrophic lateral sclerosis (ALS). The composition may also include other supporting adjutants such as bioavailable magnesium, manganese, selenium and other minerals that can support the nervous system and other vitamins and glutathione and other nutrients. Botanical extracts, urea, other supplements helpful for protection and re-enforcement of the immune system are discussed.

### CROSS-REFERENCE TO RELATED APPLICATIONS

This application is a continuation-in-part of U.S. application Ser. No. 11/932,260 (filed Oct. 31, 2007) which is a continuation-in-part of U.S. application Ser. No. 11/616,317 (filed on Dec. 27, 2006, now abandoned) which is a divisional of U.S. application Ser. No. 10/027,692 (filed on Dec. 20, 2001, now U.S. Pat. No. 7,163,709). The contents of the aforementioned patent applications are hereby incorporated by reference into this specification.

### BACKGROUND OF THE INVENTION

Superoxide is naturally produced by animals, including humans, as a response to the energy creation process and its production is accelerated in times of trauma or stress. Trauma can include exposure to radiation from on-orbit exposure or radiation treatments for cancer and other diseases, sun-burn, cuts, abrasions, bites, tooth aches and any other ache or pain. The natural oxidative process of growing old causes wrinkles and other less apparent maladies and promotes the susceptibility to diseases.

Superoxide Dismutase (SOD) is also produced by the cells to counteract the effects of an over-production of superoxide. The superoxide is broken down into hydrogen peroxide and oxygen and the animal then supplies the enzyme catalyst to break down the hydrogen peroxide into water and oxygen.

Adequate amounts of SOD can increase life span. Most people that live past age ninety tend to have unusually high levels of SOD that may relate to SOD's ability to counteract the oxidative process in animals.

SOD is also known as an anti-inflammatory treatment for traumas that include arthritis and fibrosis to name a few and is considered over 3,000 times stronger than vitamin c as a nutrient.

The symptoms of amyotropic lateral sclerosis (ALS) are well documented. The causes and treatments have been researched and studied for years with few positive treatments utilizing several different approaches. It is clear that one of the primary causes is aberrant cell production that causes a major interruption in a protective system designed to lower the risk of disease.

Superoxide is produced by cells to protect against disease and provide oxygen for normal cell function. Another component of the cell produces SOD which acts as antioxidant and is produced by mammalian species (including humans) to protect cellular components from being oxidized by too much reactive oxygen species if over-production of superoxide by cells is present as the result of aberrant cell production. There is a sensitive balance between superoxide as one of the main reactive oxygen species in the cell to protect the cell from disease and the SOD. However the aberrant genes in ALS patients cause the overproduction of superoxide in nerve cells causing disruption and nerve cell death resulting in sclerosis and the typical symptoms of ALS. It would be desirable to provide a new method to help control the balance of superoxide and SOD in a patient suffering from ALS.

### BRIEF DESCRIPTION OF THE INVENTION

This patent discusses an “artificial” SOD that can be easily applied and used for treating over-production of superoxide. The disclosed subject matter is related to a chemical composition and method of use for treatment of ALS patients. A Cu/Zn superoxide dismutase (SOD) is disclosed that neutralizes the debilitating effects suffered by individuals that are producing excessive superoxide causing the symptoms of ALS. The over expression of the cells toward manufacture of superoxide has been linked to other neural disorders (e.g. Down syndrome) and use of the disclosed composition to treat other superoxide-related diseases is also contemplated.

The invention utilizes a new technology directed at a manufacturing process and efficient application of Cu/Zn SOD based on a ligand system. The composition counteracts the effects of the over-production of superoxide by utilizing a ligand system that has the capability of permeating into the affected tissues and countering of the over-production of superoxide.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### DETAILED DESCRIPTION OF THE INVENTION

ALS is one of the most common neural muscular diseases worldwide and effects people of all races. One or two out of 100,000 people are affected each year and usually between the ages of 40 and 60 years of age. Neural muscular diseases often originate in the lower part of the brain and are the result of aberrant cells. Superoxide dismutase produced by an individual who is not affected rids any excess superoxide naturally while an ALS patient requires help.

ALS causes muscle weakness and atrophy throughout the body caused by the degeneration of the upper and lower motor neurons and the individual may lose the ability to initiate and control all voluntary movement. However it has been noted that even in advanced stages of the disease the bladder and bowels sphincter muscles and the muscles responsible for eye movement are usually spared until the terminal stages of the disease. Experiments with mice have illustrated severe pathologies evident in mice genetically engineered to lack the ability to provide superoxide dismutase according to the metabolic pathways chart (aerobic)—Krebs's Cycle. Mice lacking in the ability to provide the SOD are subject to a wide range of pathologies and demonstrate accelerated age-related muscle mass loss.

Neuro-degeneration diseases are present in individuals who have a genetic susceptibility to the diseases and certain triggers can cause the disease to activate. Recent theories include the cynobacteria neurotoxic non-protein amino acid, beta-methelamino-L-alanine (BMAA) that is produced by symbiotic cynobacteria and can be transferred up the food chain from a aquatic environment into the food chain. The presence of the protein-bound BMAA in the brains of North American patience dying with ALS and Alzheimer's disease has been confirmed (concentrations >1000 micrograms) but not in the brains of non-neurological controls or Huntington's disease.

Artificial SOD is capable of treating the deficiencies in the realm of the “normal” aerobic systems that produce SOD's and can also eliminate the anaerobic diseases present including any living disease associated with the protein-bound BMAA. It is common for bacteria other diseases to be present when sclerotic tissue forms that do not follow the Krebs cycle.

### Treatments

Presently there are several common forms of the isolated protein SODs co-factored with copper and zinc, or manganese, iron or nickel. The most common form is a copper and zinc complex commercially available in a purified form from bovine erythrocytes which are difficult to isolate and produce. There are few references to this complex in scientific papers that indicate a high level of success.

The subject matter described herein provides a manufactured ammonia ligand copper/zinc SOD that can include other essential minerals that is efficient, cost-effective and easy to apply.

### Composition

The composition for treating ALS and other neural diseases challenged by excessive superoxide production includes at least one complex cation and inorganic coordination complexes formed by the coordinate bond formation between an electropositive mineral cation (positive) and molecular groups that pose un-shared electron pairs. The formulation as manufactured provides a delivery system for moving the mineral ions to the site of the superoxide production and neutralizes the effects using a Copper/Zinc Superoxide Dismutase (Cu/Zn SOD) in combination with other remedial actions.

A method of producing a composition for treating ALS includes the activity of adding ingredients of ammonium hydrogen sulfate ((NH₄)HSO₄) with at least one mineral composition(salt) and distilled water to the mixture, agitating said mixture and diluting said mixture to a desired concentration. The activity of agitating is slow agitation process performed at a speed slow enough to reduce extreme interaction between the ingredients. The diluted mixture includes at least one ionic mineral complex encapsulated by ligand bound ammonia molecules. The diluted mixture may be combined with a pharmaceutically acceptable carrier for delivery and other ingredients as heretofore described. The other ingredients may be added at the manufacturing site or later to develop a custom made composition for a particular need.

The invention is related to a chemical composition using bioactive elements that are able to be transported through animal (including human) tissue bound to ammonia ligands carrying Copper and Zinc for example. Other minerals such as Magnesium will utilized in this ionic transport system for purposes of treating ALS or other neurological diseases. The composition can be used by itself or in combinations with other additives for the purposes of treating the disorders

### Minerals

The manufacture and use of the highly bioavailable minerals (BAMs) provide ionic elements bound in ammonia ligands for the purpose of reinforcing the supply of metal ions to boost the immune system and provide adequate elements for prevention of mineral deficiencies. BAMs can be applied topically, injection, transdermal patches, spray, subcutaneous injection and other applications commonly used in the distribution of the product

### Manufacturing Process

The manufacturing process described elsewhere in this specification produces a complex having ammonia ligand bonds was specific cations. The cations are carried by the ligand bonds and protected from being immediately bonded with the first available negative ions thus enabling free movement between and within the cells of the animal. Each of the minerals will be processed in an acid/base solution resulting in products that have a high acidity value, yet not being corrosive to living tissue. The product is corrosive to non-living biological materials such as paper, cotton, and other processed products. A noticeable concentration of reactive ammonia is also produced by the acid/base reaction. Complex cations and inorganic coordination complexes are formed that are able to move the cations in a relatively stable fashion and allow transport throughout the plant.

### Chemistry

Ammonium Sulfate and Sulfuric Acid:

Definition of terms

| | | | |
|---|---|---|---|
| 1. | Sulfuric acid | H₂SO₄ | m.w. = 98.07 g per mole |
| 2. | Ammonium sulfate | (NH₄)₂SO₄ | m.w. = 132.23 g per mole |
| 3. | Ammonium hydrogen | (NH₄)HSO₄ | m.w. = 115.10 g per mole |
| | sulfate | | |

(Ammonium Bisulfate)

The sulfuric acid and ammonia are combined in the presence of water.

| | | | | |
|---|---|---|---|---|
| H₂SO₄ + | NH₃ + | H₂O = | (NH₄)₂SO₄ + | 2H₂O |
| 98 g. | 34 g. | 36 g. | 132 g. | 36 g. |

The ammonia sulfate and water has a typical pH of 5.5-6.0. Ammonium sulfate by itself when dissolved in water does not exhibit any ammonia odor. Solid ammonium sulfate can disassociate, using one mole of ammonia and converting it to ammonium hydrogen sulfate, (NH₄)₂SO₃=NH₃+NH₄HSO₄

In the presence of water, the ammonia loss can then recombined to

NH₃+H₂O=NH₃OH or (NH₄)¹⁺+(OH)¹⁻

The ammonium bisulfate being a weakly acidic salt does not react with the diluted and weak base, ammonium hydroxide to recombine into ammonium sulfate. Ammonium hydrogen sulfate is more acidic than ammonium sulfate. Neither a solution of ammonium sulfate nor ammonium bisulfate will burn the skin on contact. Additional loss of ammonia from solid ammonium hydrogen sulfate under conditions of high heat and moisture provides the following;

NH₄HSO₄+heat+moisture=NH₃+H₂SO₄+H₂O

The result is sulfuric acid. Sulfuric acid and ammonium hydrogen sulfate are a basic building block for producing the ligand bonded cation complex hereinafter referred to as the ligand complex.

Ammonium hydrogen sulfate may be generated by two methods;

1. H₂SO₄+NH₄OH=NH₄HSO₄ (one mole of sulfuric acid and one mole of ammonium hydroxide (ammonia).

2. H₂SO₄+(NH₄)₂SO₄=2NH₄HSO₄ (one mole of sulfuric acid and one mole of ammonium sulfate).

The first reaction is very exothermic, and because the ammonia in water is not stable, ammonia fumes are generated thereby reducing the amount of ammonia available to react with the sulfuric acid. The result is a very acidic solution, having a small quantity of sulfuric acid un-reacted due to the loss of some quantity of the ammonia in the steam generated by the high exothermic reaction. This is less desirable than the second reaction that is the preferred process used to produce the composition for use.

The physical and chemical properties of the ligand complex are shown below.

Boiling point; 224.2° F.

Vapor pressure; 0.1 MM at 68° F.

Vapor density; 1.00

Solubility in water: very soluble

Appearance and odor: clear liquid—mild odor

Specific gravity: 1.35

Melting point: not applicable

Evaporation rate: not applicable

Flashpoint: none

Flammability limits: none

Stability: stable

pH: less than 1.0

### Complex Ion Formation and Ligand Bonds

The complex ions and inorganic coordination complexes are formed by the coordinate bond formation between an electro positive mineral cation (positive) and molecular groups that possesses unshared electron pairs (ammonia). Every metal ion has at least one coordination sphere which determines the number of coordinate bonds possible for each mineral atom. The coordinate bonds attract negatively charged ions possessing unshared electron pairs. All cations except groups IA and IIA (periodic table) exist as complex cations with a definite number of coordinating groups bound to them. The cations use the unshared pair in attempting to fill gaps in the outer electron orbitals where those electron shells are incomplete. The bonds formed between the cation and the unshared pair of electrons become ligand bonds.

An exemplary compound is produced as a result of the acid/base reaction when sulfuric acid is combined with ammonia sulfate described above is ammonia (NH₃) one of the products formed in the acid/base reaction. Ammonia is one of the compounds having an unshared pair of electrons that enables ligand bond formation between itself and the free cation in solution. The nitrogen molecule of the ammonia includes an unshared pair of electrons. Ammonia is very reactive in ligand binding due to its respectively small size, and the unshared pair of electrons. The three hydrogen atoms cannot equalized the charge due to repulsion between the electron pair making ammonia polar. Therefore, ammonia may enter into the following examples of complexes:

Cu⁺²(Copper ion)+4NH₃={Cu(NH₃)₄}⁺²

Zn⁺²(Zinc ion)+4NH₃={Zn((NH₃)₄}⁺²

The number of ammonia molecules is double the metallic ion valence, and that the valence charge does not change. The unshared pair of electrons forms the ligand bond, the ligand supplying both the unshared electrons. The resulting compound is a plurality of ammonia molecules ligand bonded to a single molecule of ionic zinc forming a “BAM” (encapsulated mineral(s) surrounded by ammonia “ligand bonds”).

The molecular diagram shown below is for purposes of example only and the zinc cation may be substituted by any of the cation shown in the next table.

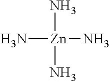

This compound including ammonia encapsulating a bioactive mineral cation is herein after referred to as a ligand complex. Additionally, urea may be included in the formulation and result in ligand bonding of the cations with the urea. In this composition, the BAM minerals could be formulated with the urea producing a product containing higher than anticipated mineral content than is normally expected in the formulations.

Examples of the ligand compounds may include, but are not limited to the following complexes shown below:

Ion-complexes with Ammonia (Ligand complexes)

| | | |
|---|---|---|
| 1. | Zinc | Zn(NH₃)₄}⁺² |
| 2. | Manganese | Mn (NH₃)₄}⁺² |
| 3. | Copper | Cu(NH₃)₄}⁺² |
| 4. | Magnesium | Mg(NH₃)₄}⁺² |
| 5. | Cobalt | Co(NH₃)₄}⁺² − Co(NH₃)₆}⁺³ + 1e⁻ |
| 6. | Chromium | |
| | Tri-valent | Cr(NH₃)₆}⁺³ |
| | Hexa-valent | Cr(NH₃)₁₂}⁺⁶ |
| 7. | Molybdenum | Mo(NH₃)₆}⁺³ |
| 8. | Selenium | Se(NH₃)₈}⁺⁴ |
| | | Se(NH₃)₁₂}⁺⁶ |
| 9. | Vanadium | V(NH₃)₁₀}⁺⁵ |

*Normally, in aqueous solutions without complexing agents, cobalt + 2 is the favored state. In the presence of complexing agents such as ammonia (NH₃), the complexes of cobalt have greater stability and are more stable in the basic than acid media.

Additionally, ligand bonding is conducive to maintain the abundance of hydrogen ions. The resulting solution has a very low pH (at or near zero) reading because of the ligand bonded mineral ions. The product does not act as a conventional acid because of the stability of the mixture of ligands. The pH of the products are not indicative of the expected acid characteristics one might imagine at a pH of 1.0 or below. The solution can only be reduced by non-heat evaporation to a certain volume.

It is possible to mix the various minerals described herein according to the process described in commonly owned U.S. Pat. No. 7,163,709. The content of U.S. Pat. No. 7,163,709 is hereby incorporated by reference.

### Secondary Active Complexes

The chemical reactions described above resolved in the mixture including a high level of sulfur which functions as a secondary active complex that operates synergistically with the ligand complex described above by providing the animal with an additional source of sulfur, a known disease treatment. The high level of free sulfur is able to be transported to the various locations in the biological system by normal mode of transport and works to speed reconstitution of damaged tissue and build new cells. Sulfur is a non-metallic acidic macro mineral usually consumed as part of a larger compound (Zinc, Copper, etc.). The benefits of sulfur in animals is well known and previously described.

### Method of Manufacture

An exemplary method of producing the composition having a ligand complex is described herein. Ammonium hydrogen sulfate (NH₄HSO₄) is prepared as described above and is used in the concentrated form. The ammonium hydrogen sulfate is added to distilled water. The minerals added which are dissociated to become the bioavailable ionic mineral ligand complex are added in their respective sulfate forms, e.g. Zinc sulfate, copper sulfate, magnesium sulfate, etc. any of the minerals listed above may in their sulfate formed be mixed with the ammonium hydrogen sulfate and distilled water to produce the ligand complex. Preferably, the mineral's salt purity is manufacturing or pharmaceutical grade with no mineral impurities. The product destined for utilization in human and animal treatments must be of pharmaceutical grade with no impurities. The processing of the minerals to form the product is only a general guideline and the concentration of the minerals in the solution can be adjusted to produce custom-made formulations to suit any particular neurological disorder such as ALS.

An exemplary composition for making 1 gallon of the nine (9) mineral complexes listed above involves preparing a mix, under agitation, slowly adding the combinations to prevent an exothermic reaction of the following ingredients:

98 ounces of water (H₂O)

14 ounces of ammonium hydrogen sulfate (NH₄HSO₄)

16 ounces (one of the nine listed) mineral salts alone or in combination(s) with other minerals on the list to make a total of 16 ounces of mineral.

The exemplary composition may include, for example, 16 ounces of zinc sulfate; or 8 ounces of zinc sulfate and 8 ounces of copper sulfate; or 8 ounces of zinc sulfate, 4 ounces of copper sulfate and 4 ounces of magnesium sulfate. Any sulfate form of the minerals listed above are the preferred salts but other salts can be used and may be combined in any ratio to equal 16 ounces. The composition may include any number of minerals sulfate complexes in order to produce a composition having a number of ligand complexes equal to the number of different minerals in the minerals sulfate complexes added during mixing. The description of the mineral composition in the sulfate form is for purposes of example only and any other form of mineral composition including but not limited to oxides, carbonates, nitrates and others may also be used to produce the composition. The activity of the composition would not be considered exemplary however.

A solution containing a mix of the prepared mineral(s) may contain only one of the minerals and additional supplements or all of the mentioned minerals plus selected supplements to achieve the desired effect. However, zinc and a small amount of copper will help balance (Mineral Inter-relationships) the effect of the possibility of too much zinc and other supplements. Other minerals such as magnesium, manganese, selenium. and chromium in proper balance may be required to achieve control of the over production of superoxide and aid in the healing of the neurons and genesis of the process at the heart of the disease.

All the minerals will act independent of one another much as gases do in Dalton's Law and be assimilated by the tissues on an as needed basis. For example, copper and zinc are required in the Cu/Zn SOD. This combination in the Cu/Zn SOD is critical in the treatment of ALS while the other ionic minerals can be utilized to repair damaged cells and rejuvenate damaged neurons. Other necessary minerals for neural health and function that were present in the past may have been oxidized by the superoxide being produced and producing oxides and other combination that can become sclerotic in nature. The high production of the SOD along the spine is in effect creating an environment that can be treated utilizing a Cu/Zn SOD that will neutralize the effect. Other minerals can be utilized that are directed at the repair and enhancement capabilities of the neurons and supporting tissues.

It is highly important to understand the delicate balance of mineral interactions to prepare formulations that meet individual needs to be incorporated. Factors include the rate of Superoxide produced by the patient given a profile of physical activity and a norm established. Generic formulations can be constructed for general applications but this patent discusses the possibility of custom formulations utilizing several methods of application directed at slow release at appropriate dosage for immediate relief and long term availability utilizing slow release systems. Milligrams per kilograms body weight, age, sex of the individuals will all be taken into account.

Two minerals must be formulated into the BAM formulation at the right proportions and counter act the influence of one against the other. For example the copper and zinc are in proportions that act to counterbalanced each other physiologically on a basis of 7:2 (zinc to copper) and with the ammonia (NH₃) to form of ligand complex. The ligand travels through the selective membrane (epidermis) and travels through the patients system to the point of the excessive Superoxide production (the spine and associated areas for ALS for example). The product will penetrate the area with the Cu/Zn SOD to counter-act the excessive superoxide and attack any non-Krebs Cycle disease in that area.

Animals form SODs (Metabolic Pathways) in normal tissues. The mistakes in the genes or other factors create a tremendous overload of the animal system with superoxide and lead to major complication including atrophy and sclerosis. An abnormal cycle will result from the production of excessive Superoxide. However once the disease is properly diagnosed relief utilizing this technology is possible.

The formulations discussed in this patent will be incorporated into the SOD on an as needed basis attached to the mineral complex to make a Cu-SOD, a Zn-SOD, or a Cu/Zn-SOD. The enzyme superoxide dismutase catalyzes dismutation of superoxide into oxygen and hydrogen peroxide. Therefore, it is an important antioxidant defense in almost all cells exposed to oxygen. The SOD catalyzed dismutation of superoxide may be written using the following half reactions:

M⁽ⁿ⁺¹⁾+SOD+O₂⁻=M⁽ⁿ⁺⁾+SOD+O₂

M⁽ⁿ⁺⁾+SOD+O₂⁻=2H⁺=M⁽ⁿ⁺¹⁾+SOD+H₂O₂

Where M may be, but is not limited to:

a) Cu (n=1); (b) Zn (n=2); (c) Mn (n=2) Fe (n=2); (e) Ni (n=2).

In this reaction the oxidation state of the metal cation oscillates between n and n+1.

### Naturally Occurring SODs

Several common forms of SOD exist naturally and are proteins co-factored with copper (Cu) and zinc (Zn), manganese (Mn), iron (Fe) or nickel (Ni). Cytosols of almost all eukaryotic cells containing SOD's are combined with copper and zinc (Cu—Zn-SODs). The Cu—Zn SOD and design is a homodimer of molecular weight of approximately 32,500. The Cu and Zn are joined primarily by hydrophobic and electrostatic interactions. The ligand complexes of copper and zinc are histidine side chains where as the ligand complexes of manganese ions are 3 histidine side chains. The incorporation of the ligand complex into the SOD enables the ligand complex to travel throughout the animal in a protected form and without compromising the effectiveness of the ligand complex. Once the SOD with the ligand complex reaches a target cell the cation within the ligand complex is released into the cell.

However the natural forms are difficult to purify and are expensive while the artificial SOD can be produced in large quantities and utilized in several application technologies such as creams, injectables, transdermal patches and other methods currently used in current practice.

### Handling

The resulting formulation can be prepared in many ways for application and will vary with the intended use. Formulations prepared for treatment of ALS and other neurological animal diseases (including humans) suffering from mineral deficiency will require selection of the proper formulation containing the necessary minerals. The formulations are stable at a pH near or below pH 1.0 in a wide variety of carriers. However, the active composition is prepared using the liquid. Additionally, the low pH of the composition may be diminished if the composition crystallizes.

The mineral cation ingredients in the active composition will vary in proportion depending on the intended use and be added in certain formulation depending on the type and purpose thereof. The added inserts used in the formulation will also vary considerably depending on the site and purpose of the application. Other active compounds may be added if the proposed components prove to be beneficial to the formulation. However, the basic ingredients are known to be effective without any additional components.

Additional elements may be added to the composition that aid in the overall effectiveness of the formula. For example, a structured use of the proper vitamins and other supplements prescribed will be incorporated and have demonstrated additional effectiveness for the treatment.

Additives will included on a as-needed basis and include inserts such as surfactants including dimethyl sulfoxide (DMSO), urea based compounds, detergents to aid in the penetration through the skin and/or for other reasons necessary in the manufacture of formulations for injection, suppositories, transdermal-patches, beads for injection and other methods of depositing the product in the vicinity of the trauma.

Treatment of the diseases and/or application for the purpose of promoting general health may vary and will be, but not limited to be administered primarily as a topical application, taken orally, given by injection, transdermal patch, implant or by the use of methods of treatment discussed such as application of the formulation through, or incorporated into and/or as a gel cap, tablet, powder, food additive, drops liquid, beverage, rinse, gargle, pill, capsule, lozenge, cough drop, transdermal patch, ointment salve, cream, lotion, gel, intravenous drip, and/or adapted for periodical administering a therapeutically effective dosage of at least one of topically, orally, parenterally, intravenously or subcutaneously. The ligand complex composition has a low pH (less than 1.0) and the low pH must be maintained when forming any of the above formulations.

### Discussion

It will be understood that each of the elements described above or two or more elements together may also find a useful application in other types of methods differing from the methods described above.

Since the invention is described with reference to different embodiments and pointed out in the annexed claims, and since numerous modifications and changes may become readily apparent to those skilled in the art after reading this disclosure, it should be understood that we do not wish to limit the scope of the overall invention to the exact composition, or method of making same, described above and claim below, since it is understood that various omissions, modifications, substitutions and changes in the form and details of the invention and its application can be made by those skilled in the art without departing in any way from the spirit of the present invention.

Without further analysis, the foregoing will so fully reveal the just of the present invention so that others can, by applying current knowledge, readily adapt it for various applications without omitting features that, from the standpoint of prior art, fairly constitute essential characteristics of the generic or specific aspects of this invention.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating a disease or other trauma, the method comprising:
administering to a human patient suffering from a trauma including a disease with an ionic mineral complex comprising:
an ionic metal bonded to a plurality of ammonia ligands to form a metal-ligand complex, the ammonia ligands enabling transport of the metal-ligand complex through the human patient to a region affected by over-production of superoxide.

2. The method as recited in claim 1, wherein the ionic metal is a cation selected from the group consisting of zinc, manganese, copper, magnesium, cobalt, chromium, molybdenum, selenium, vanadium and combinations thereof.

3. The method as recited in claim 1, wherein the ionic metal is a cation selected from the group consisting of zinc, copper and combinations thereof.

4. A method for treating a neural disease, the method comprising:
a. identifying a human patient that is suffering from amyotrophic lateral sclerosis (ALS);
b. administering to the human patient suffering from a neural disease an ionic mineral complex comprising of a first ionic metal bonded to a first plurality of ammonia ligands to form a first metal-ligand complex and the first plurality of ammonia ligands enabling transport of the first metal-ligand complex through the human patient to a region affected by over-production of superoxide.

5. The method as recited in claim 4, wherein the ionic mineral complex further comprises an additive selected from the group consisting of a surfactant, di-methyl sulfoxide (DMSO), a urea-based compound, a detergent, a hygroscopic compound, and any combination thereof.

6. The method as recited in claim 4, wherein the ionic mineral complex further comprising an additive selected from the group consisting of urea, dimethyl sulfoxide (DMSO), a surfactant, a detergent, and any combination thereof.

7. The method as recited in claim 4, wherein the ionic mineral complex is in the form of a topical application, an injectable, a suppository or a transdermal patch.

8. The method as recited in claim 4, wherein the step of administering comprises topical administering, oral administering, intravenous administering, suppository administering or subcutaneous injection administering.

9. The method as recited in claim 4, wherein the step of administering comprises topical administering using a transdermal patch.

10. The method as recited in claim 4, wherein the step of administering comprises topical administering using a cream.

11. The method as recited in claim 4, wherein the step of administering comprises administering using a gel cap, a tablet, a powder, a food additive, a liquid, or an oral rinse.

12. The method as recited in claim 4, wherein the ionic mineral complex has a pH of less than 1.

13. The method as recited in claim 4, wherein the ionic mineral complex comprises two or more different ionic metals.

14. The method as recited in claim 4, wherein the first ionic metal is copper and wherein the ionic mineral complex further comprises a second ionic metal bonded to a second plurality of ammonia ligands to form a second metal-ligand complex, the second plurality of ammonia ligands enabling transport of the second metal-ligand complex through the human patient to the region affected by over-production of superoxide.

15. The method as recited in claim 14, wherein zinc and copper are present in a ratio of about 7:2.

16. The method as recited in claim 14, wherein the second ionic metal is selected from the group consisting of manganese, magnesium, cobalt, selenium, chromium and vanadium.

17. A method as recited in claim 14, wherein the ionic mineral complex further comprises a carrier specific for a predetermined trauma.
